(19) United States
(12) Patent Application Publication
(10) Pub. No.: US 2019/0117157 A1
(43) Pub. Date: Apr. 25, 2019

(54) SENSORS FACILITATING MONITORING OF LIVING ENTITIES

(71) Applicant: International Business Machines Corporation, Armonk, NY (US)

(72) Inventors: Huan Hu, Yorktown Heights, NY (US); John Knickerbocker, Monroe, NY (US); Katsuyuki Sakuma, Fishkill, NY (US)

(21) Appl. No.: 15/793,452

(22) Filed: Oct. 25, 2017

(51) Int. Cl. A61B 5/00; G02B 5/18; G01B 11/16; G01B 7/16; G01K 3/10; A61B 5/01; A61B 5/1455

(52) U.S. Cl. CPC A61B 5/6832; G02B 5/1809; G02B 5/1857; G01B 11/16; G01B 7/22; G01K 3/10; A61B 2562/12; A61B 5/01; A61B 5/14552; A61B 5/0022; A61B 2562/0266; A61B 2562/0285; A61B 5/6826

(57) ABSTRACT

Apparatus, systems, and methods of manufacture of sensors facilitating monitoring of living entities. In one example, a system comprises a flexible substrate comprising an adhesive adapted to cause the flexible substrate to adhere to a defined surface. A silicon substrate or film can be disposed on the flexible substrate, wherein the silicon substrate or film is formed to include one or more nanogratings adapted to receive and reflect light based on strain on the defined surface.

### BACKGROUND

The subject disclosure relates to sensors, and more particularly sensors facilitating monitoring of living entities.

### SUMMARY

The following presents a summary to provide a basic understanding of one or more embodiments of the invention. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, apparatus and/or methods that facilitate production of integrated sensors facilitating monitoring of living entities are described. In some embodiments, the sensors are fingernail sensors although in other embodiments, the sensors can be skin sensors, hair sensors or any number of other types of sensors.

According to an embodiment, a system is provided. The system can comprise a flexible substrate comprising an adhesive adapted to cause the flexible substrate to adhere to a defined surface. The system can also comprise a silicon substrate or film disposed on the flexible, wherein the silicon substrate or film is formed to include one or more nanogratings adapted to receive and reflect light based on strain on the defined surface.

According to yet another embodiment, a method is provided. The method can comprise providing a flexible substrate comprising an adhesive adapted to cause the flexible substrate to adhere to a defined surface, wherein the flexible substrate is comprised of a polymer. The method can comprise depositing silicon on the flexible substrate. Additionally, the method can comprise forming one or more nanogratings on the silicon, wherein the one or more nanogratings are adapted to receive and reflect light based on a strain or a deformation of the defined surface.

According to another embodiment, a device is provided. The device can comprise a polymer substrate comprising an adhesive adapted to cause the polymer substrate to adhere to a defined surface. The device can also comprise a silicon substrate or film disposed on the polymer substrate, wherein the silicon substrate or film comprises one or more nanogratings adapted to receive and reflect light based on the pressure on the defined surface.

According to an embodiment, a system is provided. The system can comprise a flexible substrate comprising an adhesive adapted to cause the flexible substrate to adhere to a defined surface. The system can also comprise a silicon substrate or film disposed on the flexible, wherein the silicon substrate or film is formed to include one or more nanogratings adapted to receive and reflect light based on strain on the defined surface. Additionally, the system can comprise a mobile device configured to receive strain data associated with a strain experienced by the flexible substrate.

According to another embodiment, another device is provided. The device can comprise a polymer substrate comprising an adhesive adapted to cause the polymer substrate to adhere to a defined surface. The device can also comprise a silicon substrate or film disposed on the polymer substrate, wherein the silicon substrate or film comprises one or more nanogratings adapted to receive and reflect light based on the pressure on the defined surface. Furthermore, the device can comprise a collimating lens adapted to receive the reflected light from the one or more nanogratings.

These and other features will become apparent from the following detailed description of illustrative embodiments thereof, which is to be read in connection with the accompanying drawings.

### DESCRIPTION OF THE DRAWINGS

FIG. 1 illustrates a non-limiting side view of an integrated strain sensor that facilitates monitoring of living entities and having a photonic crystal structure in accordance with one or more embodiments described herein.

FIGS. 2A, 2B, and 2C illustrate non-limiting side views of a spalling process to fabricate the integrated sensor of FIG. 1 in accordance with one or more embodiments described herein.

FIG. 3A illustrates a non-limiting fabrication process flow to fabricate of the integrated sensor of FIG. 1 in accordance with one or more embodiments described herein.

FIG. 3B illustrates a block diagram of a strain sensor system that facilitates monitoring of living entities in accordance with one or more embodiments described herein.

FIG. 4 illustrates a non-limiting two-dimensional photonic crystal sensor and one-dimensional photonic crystal sensor in accordance with one or more embodiments described herein.

FIG. 5 illustrates a non-limiting system that facilitates monitoring of a living entity in accordance with one or more embodiments described herein.

FIG. 6 illustrates a non-limiting top view of the integrated sensor of FIG. 1 in accordance with one or more embodiments described herein.

FIG. 7A and 7B illustrate non-limiting spectrum peak shift graphs due to a strain on the integrated photonic, crystal structure of FIG. 1 in accordance with one or more embodiments described herein.

FIG. 8 illustrates a flow diagram of another example, non-limiting method that facilitates fabricating an integrated sensor in accordance with one or more embodiments described herein.

FIG. 9 illustrates a flow diagram of another example, non-limiting method that facilitates fabricating an integrated sensor in accordance with one or more embodiments described herein.

FIG. 10 illustrates a flow diagram of another example, non-limiting method that facilitates fabricating an integrated sensor in accordance with one or more embodiments described herein.

FIG. 11 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

It is to be understood that the present disclosure can be described in terms of a given illustrative architecture comprising nail sensor fabrication; however, other architectures, structures, materials and process features and steps can be varied within the scope of the present invention.

It should also be understood that when an element such as an interface layer, load, etc. is referred to as being “on” or “over” another element, it can be directly on the other element or intervening elements can also be present. In contrast, when an element is referred to as being “directly on” or “directly over” another element, there are no intervening elements present. It should also be understood that when an element is referred to as being “connected” or “coupled” to another element, it can be directly connected or coupled to the other element or intervening elements can be present. In contrast, when an element is referred to as being “directly connected” or “directly coupled” to another element, there are no intervening elements present.

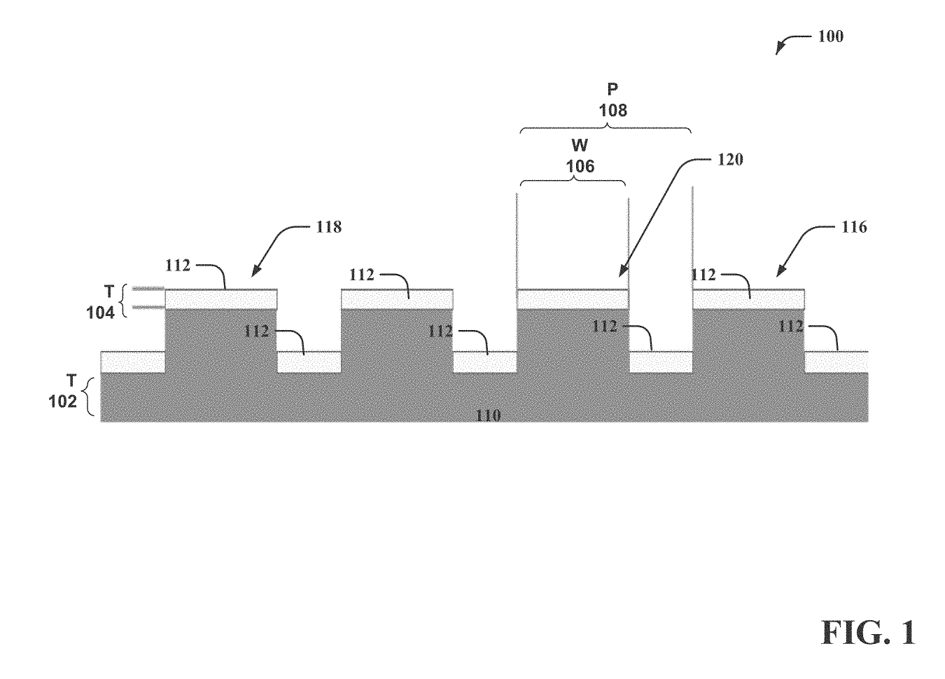

FIG. 1 illustrates a non-limiting side view of an integrated strain sensor that facilitates monitoring of living entities and having a photonic crystal structure in accordance with one or more embodiments described herein. The integrated sensor can be or include strain sensor 100 as shown. In various embodiments, the strain sensor 100 can be made from a photonic crystal formed from a silicon (Si) substrate 110 and a metal titanium dioxide (TiO₂) 112 material. In some embodiments the photonic crystal can he formed from silicon nitride, silicon oxide, and/or glass that has a first optical property matching a second optical property of a polymer layer on an etched silicon substrate. In other embodiments, the Si substrate 110 thickness Cr) 102 can be between 200 nanometers (nm) to 50 microns (pm), and the TiO₂ 112 material thickness 104 can between 10 nm to 2 μm. While the various ranges are provided, in other embodiments, other ranges and/or values can be employed.

The width (W) 106 of the metal TiO₂ 112 can be 10 nm to 2000 nm. Additionally, the pitch (P) 108 (e.g., metal width plus metal spacing) can be 200 nm to 2000 nm. The silicon nanogratings 116, 118, 120 spalled out from the photonic crystal substrate (e.g., Si substrate) 110 can be integrated with a polymer (not shown). In some embodiments, the silicon nanogratings 116, 118, 120 can be formed in the strain sensor 100 via a semiconductor manufacturing process. For example, a specific thickness of titanium oxide can be deposited onto silicon substrate 110 to create a photonic crystal. Due to the periodicity of a nanograting, the wavelengths associated with the nanogratings can be similar to the wavelengths of visible optical light. Thus, light can interact with the nanogratings (e.g., nanogratings 116, 118, 120) to create oscillations (e.g., increases in signal-to-noise ratio) and produce a peak in wavelength when the surface of the body on which the strain sensor 100 is placed bends. The peak in wavelength can be due to the strain on the surface of the body (e.g., surface of the fingernail) when the strain sensor 100 is placed on the body (e.g., placed on the fingernail).

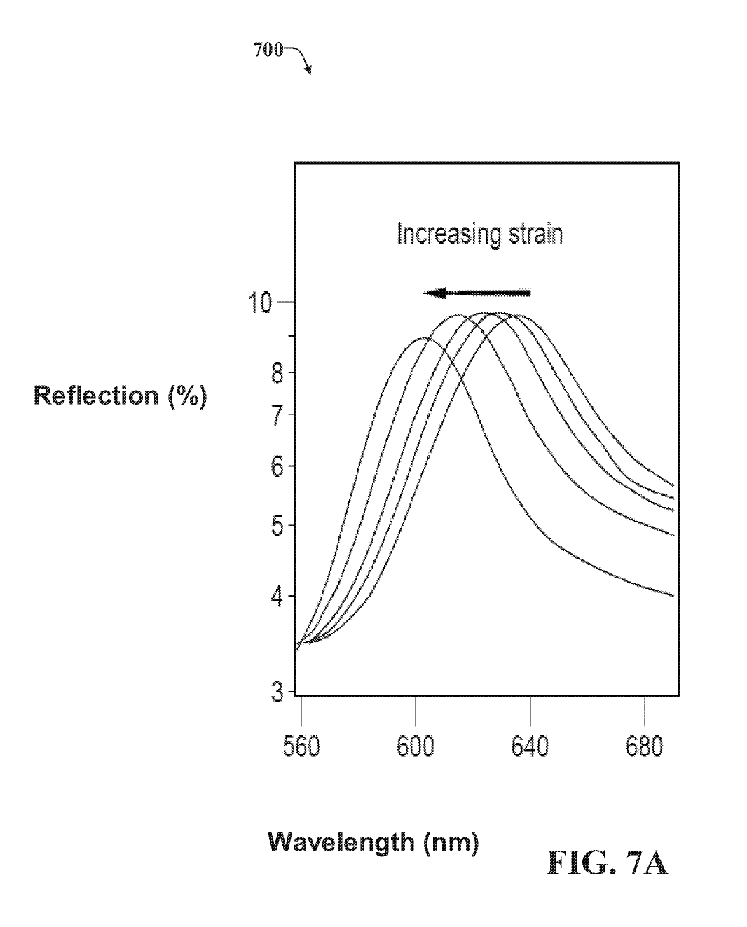

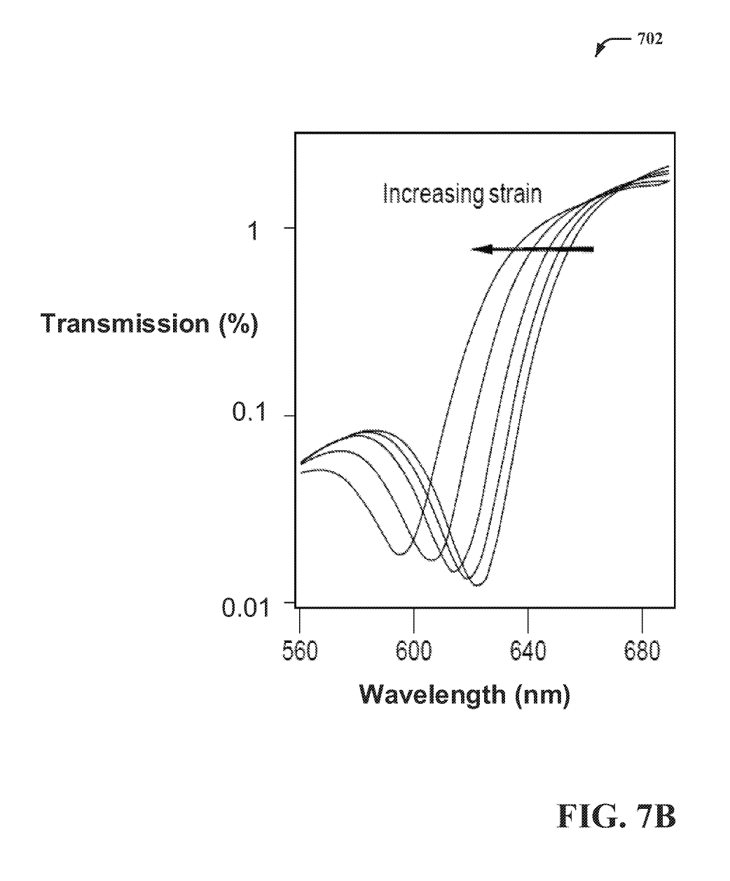

In some embodiments, a surface of the strain sensor 100 can be coupled to the fingernail of a subject (e.g., human, animal). In some embodiments, the strain sensor 100 can be less than 200 microns (um) glued on the surface of the subject. The strain sensor 100, can be applied to any number of surfaces associated with a subject, including, but not limited to, hair, skin or the like. The strains or deformation of the surface of the body or hair (e.g., fingernail) can cause an optical grating structure change, resulting in an optical spectrum peak shift (as shown and described with reference to FIGS. 7A and 7B). The optical spectrum peak shift can be measured and the measurement can be employed to determine or predict medical or health outcomes related to the subject.

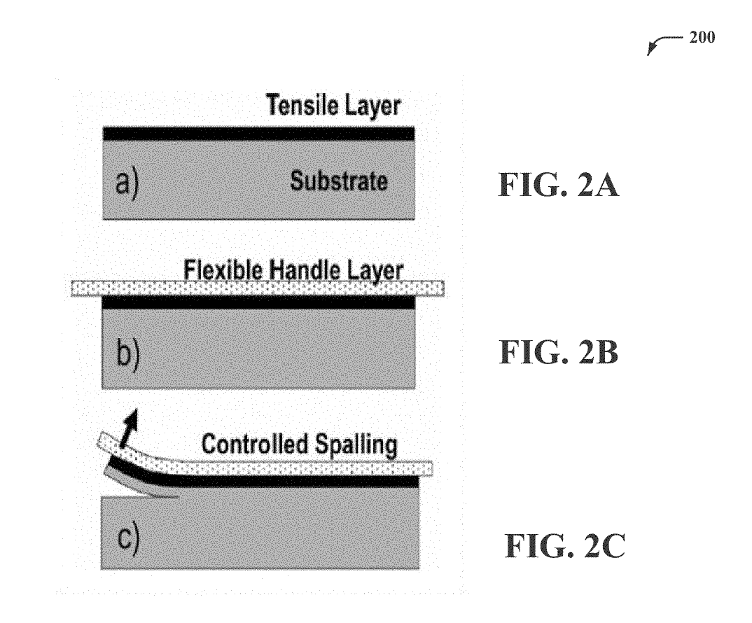

FIGS. 2A, 2B, and 2C illustrate non-limiting perspective views of a spalling process to fabricate the integrated sensor of FIG. 1 in accordance with one or more embodiments described herein. Repetitive description of like elements employed in other embodiments described herein is omitted for sake of brevity.

In some embodiments, a spalling process can be employed to separate the nanogratings 116, 118, 120 from the Si substrate 110 in a controlled manner In other embodiments, other processes can be employed.

Spalling is the process of separating pieces of material from a larger solid body and can be produced by a variety of mechanisms. As depicted in FIG. 2A, a tensile layer can be disposed upon a substrate. A flexible handle layer can be disposed upon the tensile layer as shown in FIG. 2B. As shown in FIG. 2C, the flexible handle layer can be used to separate the tensile layer and a top layer of the substrate by removing the flexible handle in a controlled fashion, resulting in spalling. The spalling process can be used to decrease the thickness 102 of the Si substrate 110 to comply with the specifications for a particular application. FY various embodiments, the spalling process can be employed to decrease the thickness of Si substrate 110 to result in a strain sensor 100 that is suitable for the surface of a fingernail, hair strand or the like.

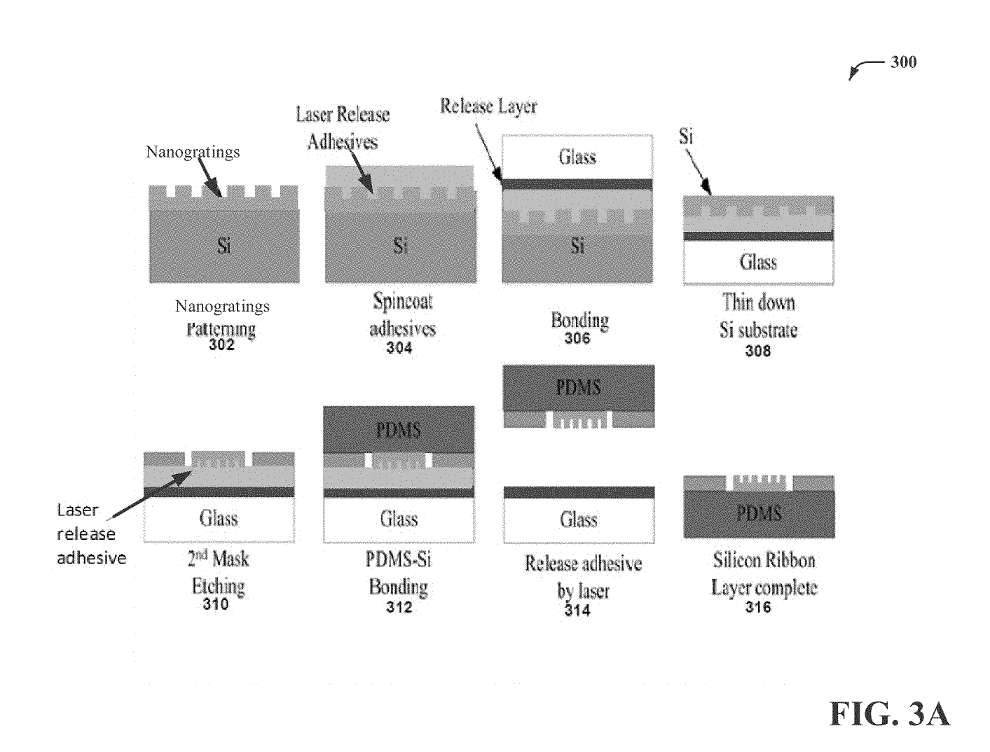

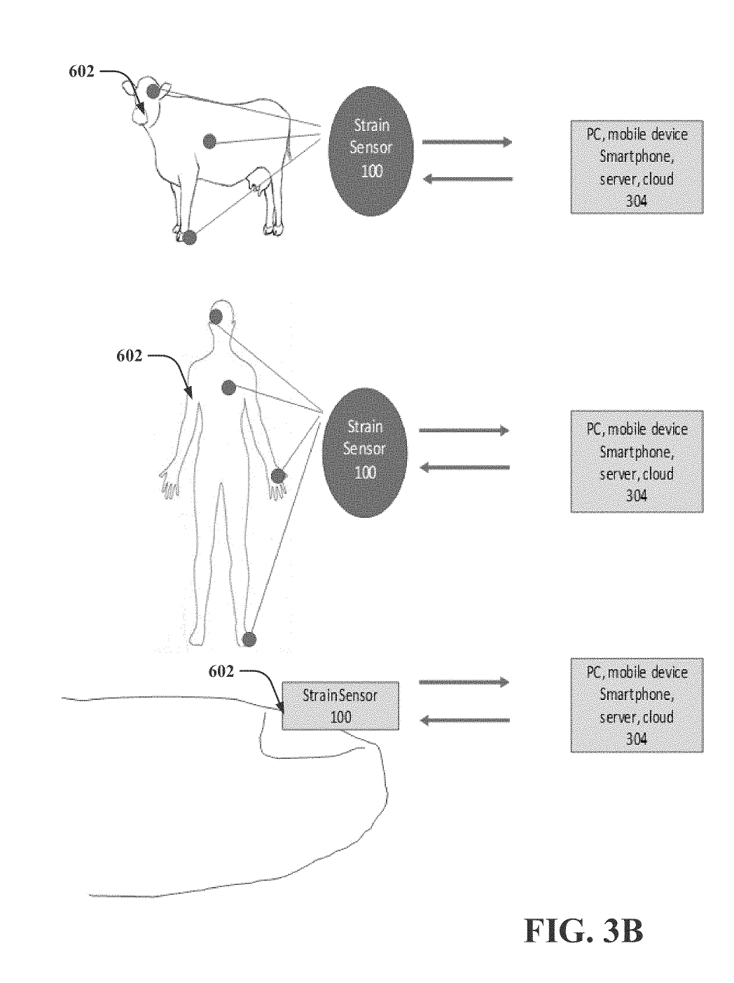

FIG. 3A illustrates a non-limiting fabrication process flow to fabricate an integrated sensor (e.g., photonic crystal sensor) that can monitor living beings in accordance with one or more embodiments described herein. FIG. 3A depicts a temporary bonding-de-bonding process flow. This can be used to fabricate a thin Si (flexible/bendable) sensor (including strain gauge and strain sensor using nanogratings). The process of FIG. 3A applied to FIG. 1 can also result in the strain sensor 100. For example, in some embodiments, the silicon ribbon layer complete step 316 of FIG. 3A can show a strain sensor 100 that can be employed in one or more embodiments described herein. Repetitive description of like elements employed in other embodiments described herein is omitted for sake of brevity.

As shown in FIG. 3A, the strain sensor (shown at step 316) can be fabricated via another process different from the spalling process depicted in FIG. 2. For this embodiment, the Si substrate can be 5-50 μm thick, so the Si substrate can be thinned down by the following process: temporary attachment to a handle wafer, coarse and fine grinding, a chemical-mechanical polish, and a removal from the handle wafer. The Si substrate can be thinned to a suitable dimension for the embodiments described herein. Generally wafers are about 730 and 780 μm for 200 mm and 300 mm diameter wafers, respectively. However, for this application, the wafer can be processed down to a 5-50 μm thickness to create the nanogratings (which can be analogous in structure, composition and/or functionality to nanogratings 116, 118, 120). The nanogratings can also be fabricated using a deep ultraviolet (UV) lithography or electron beam lithography, or nano-imprint or other nanofabrication technology to create structures of around 400-800 nm, which is below normal UV microlithography. During these steps, the Si substrate can be held to a glass or silicon handle wafer, bonded to it temporarily, then provided through a grinding process. The grinding process can comprise a course grind, a fine grind, and then a polish or chemical-mechanical polish. The structure can then be released using a de-bonding process (e.g., laser de-bonding, chemical de-bonding, thermal de-bonding, mechanical de-bonding, etc.).

Silicon nanogratings can be formed on the Si substrate via a nano lithographic step and etching process at step 302. At step 304, a laser release adhesive can be layered above the nanogratings and the Si substrate prior to a release layer being placed above the laser release adhesive at step 306. A glass layer can also be disposed on the release layer during a bonding process at step 306. At step 308, the Si substrate can be trimmed down to the aforementioned thickness. At step 310, the Si substrate can be etched during a masking process prior to being bonded to a polydimethylsiloxane (PDMS) or other polymer compound at step 312. Once the Si substrate is bonded to the PDMS, the adhesive can be removed separating the release layer and the glass layer at step 314, leaving a complete silicon ribbon layer at step 316. The resultant apparatus can be similar in structure and/or functionality to the strain sensor 100 of FIG. 1. In some embodiments, the sensor can also be a photonic crystal sensor. Alternative processes to create the nanogratings are also possible such as nano-imprinting and/or a nano-printing step for a resist and etching process.

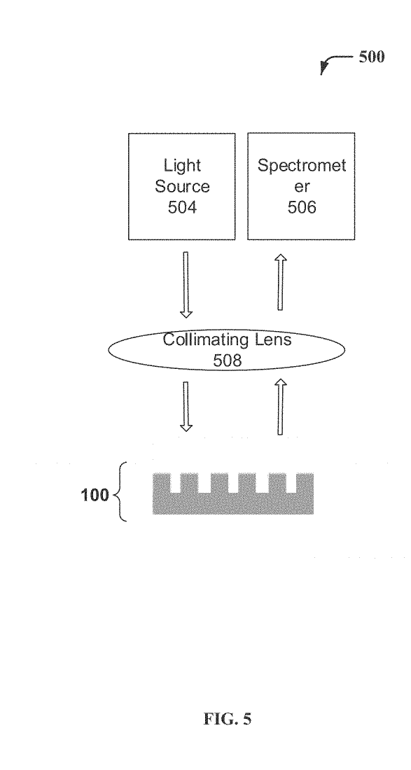

FIG. 3B illustrates a block diagram of a strain sensor system that facilitates monitoring of living entities in accordance with one or more embodiments described herein. Repetitive description of like elements employed in other embodiments described herein is omitted for sake of brevity. As shown in FIG. 3B the strain sensor 100 can monitor the of a living entity 602 and communicate this information to a mobile device 318. After accumulating data from the living entity 602, the strain sensor 100 can communicate the information to the mobile device 318 (as described in further detail with regards to FIG. 5). In some embodiments, the communication can be wireless according to various communication protocols including, but not limited to: Bluetooth, cellular, wireless fidelity (Wi-Fi), etc. In various other embodiments, the strain sensor 100 can comprise a power source, microcontroller, a battery, an antenna, and/or the ability to receive data and power, and transmit data (e.g., encrypted data).

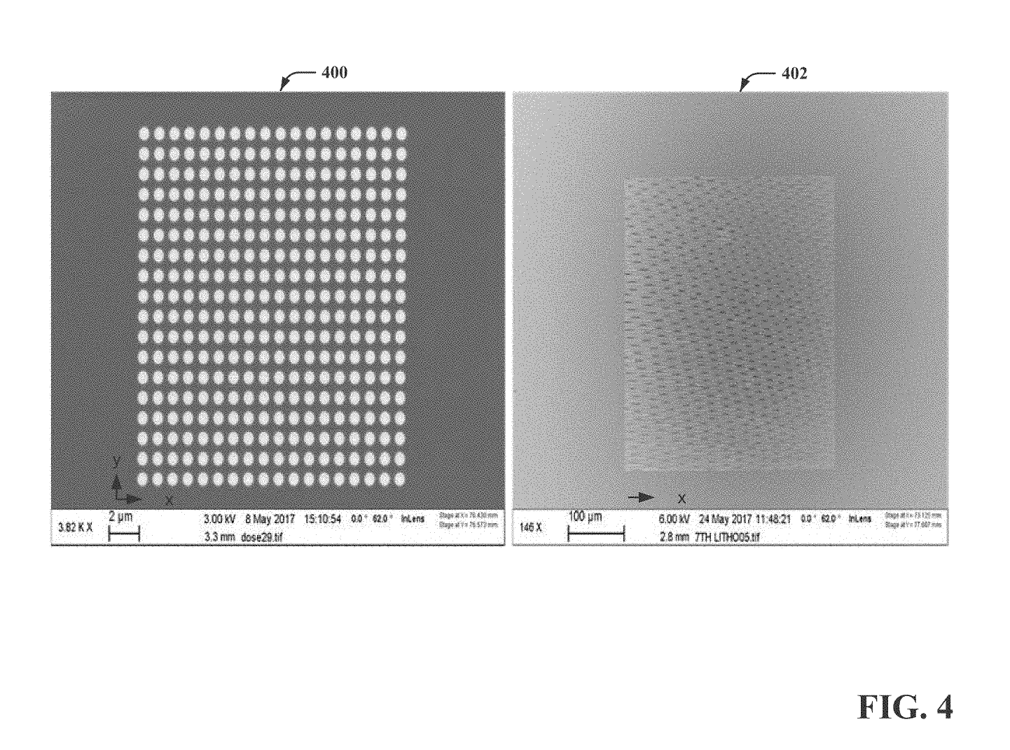

FIG. 4 illustrates a non-limiting one-dimensional photonic crystal sensor 402 and a two-dimensional photonic crystal sensor 400 (such as that formed via the process of FIG. 3 or FIG. 2) in accordance with one or more embodiments described herein. Repetitive description of like elements employed in other embodiments described herein is omitted for sake of brevity. The two-dimensional view 400 of photonic crystal sensor gratings can be an entire two-dimensional array. In some embodiments, the photonic crystal sensor gratings can be a groove type as shown by the one-dimensional photonic crystal sensor 402.

FIG. 5 illustrates a non-limiting system that facilitates monitoring of a living entity in accordance with one or more embodiments described herein. Repetitive description of like elements employed in other embodiments described herein is omitted for sake of brevity.

The integrated sensor system 500 can comprise a light source 504, a spectrometer 506, a collimating lens 508, and a photonic crystal on an integrated circuit (which can form the strain sensor 100). In some embodiments, the sensor can be a fingernail sensor that is applied to the surface of a fingernail of a human, animal and in other embodiments the sensor can be applied to other surfaces of the body (e.g., skin, hair, etc.).

The integrated sensor system 500 can detect a strain on the surface of a target area (e.g., on a fingernail of a user) based on the deformation of the target area bending, stretching or compressing. The integrated sensor system 500 can send a wireless signal to a mobile device 618, PC, tablet, etc., by using wireless communication protocols such as Bluetooth, Wi-Fi, etc. in response to detecting the strain (or in response to detecting a defined amount of strain or detecting strain meeting some other defined condition). In another embodiment, the integrated sensor system 500 can also be physically coupled to the external device (e.g., mobile device, etc.) in order to enable the system 500 to communicate directly with the aforementioned external devices. The strain sensor 100 can also be adapted to send or receive medical information associated with the user to/from an external device.

The silicon nanogratings spalled out from the processes depicted by FIGS. 2 and 3 can be coupled to the fingernail, and the strain on the fingernail can cause an optical grating structure change, resulting in light emanating from the light source 504 to be reflected in a defined manner and captured/measured/read by the spectrometer. In some embodiments, although not shown, the spectrometer can be replaced with a mobile device 618 (e.g., smart phone) that can have an installed computer application that can enable the reflected light from the nanogratings to be read by the application. The strain in the fingernail (or other body surface) can therefore result in an optical spectrum peak shift such as that shown and/or described in FIGS. 7A and 7B.

An adhesive such as glue can be one example of a substance used to facilitate adherence of the sensor to the fingernail, although any adhesive or other mechanism for chemically or physically coupling the sensor to the fingernail can be employed. In some embodiments, the strain on the fingernail can be associated with a pressure that the fingernail is experiencing. For example, in one embodiment, the light source 504 can transmit light through the collimating lens 508. The transmitted light can then pass through the collimating lens 508 and interact with the nanogratings of the strain sensor 100. The interaction of the light with the nanogratings can be reflected back through the collimating lens 508 to the spectrometer 506 and the spectrometer can measure the optical spectrum peak shift, which can be indicative of an amount and/or type and/or location of strain on the fingernail. It should be understood that the spectrometer 506, collimating lens 508, and light source 504 can be attached to the strain sensor 100 as a single system 500 or, in some embodiments, can be separate components from the strain sensor 100. For example, the spectrometer 506 can be integrated with a mobile device 618 (not shown) to wirelessly receive the reflected light via the mobile device 618. Alternatively, the spectrometer 506 can be connected to the strain sensor 100 via an optical cable.

Additionally, in another alternative embodiment, a system (not shown) can be formed to include the strain sensor (e.g., strain sensor 100 of FIG. 1) comprising a thin photodetector that can be added as a layer above the surface of the nanogratings (e.g., 116, 118, 120 of FIG. 1). Photodetectors (e.g., photodiodes, photo transistors, etc.) can sense light or other electromagnetic energy and can comprise a p-n junction that converts light photons into current. The p-n junction can be covered by an illumination window, usually having an anti-reflective coating. The absorbed photons make electron-hole pairs in the depletion region. Thus, the photodetector is operable to receive the light from the light source 504. Consequently, when the light (e.g., light emitting diode (LED) light) from the light source 504 meets the surface of the nanogratings 116, 118, 120, the light can be reflected back to the photodetector.

In yet another embodiment, the nanogratings can comprise various other sensors. For example, an optical sensor can be used to detect temperature change. The optical sensor can be a floating structure not directly attached to the body surface (e.g., fingernail, etc.). The optical sensor can also be coupled to a transmitter that can send data from the sensor that is associated with an optical spectrum peak. However, in this embodiment, thermal expansion of the optical sensor can signal a change in temperature.

Alternatively, a temperature sensor can also be used as a backup to provide a body temperature of a living entity. The optical sensor can also comprise a photoplethysmogram (PPG) (for obtaining a volumetric measurement of the heart or other organ) to measure pulse waves from the fingernail and oxygen in the bloodstream. For instance, the PPG can comprise a pulse oximeter, which illuminates the fingernail and measures changes in light absorption. A conventional pulse oximeter can monitor the perfusion of blood to the fingernail or other body parts. This sensor can be correlated with an electronic stethoscope, or an electrocardiogram measurement for the heart pulses. Additionally, the strain sensor 100 can also comprise a capacitive sensor that can be developed to prevent leakage current or losses from battery use.

The strain sensor 100 can comprise the optical nanogratings 116, 118, 120, a local electronic microcontroller, a battery, and a device that can transmit information. The strain sensor 100 can also be configured to transmit data or encrypted data to an external reader, a power station that is remote from the strain sensor 100, and/or a system attached to a living entity. In various embodiments, the electronics of the strain sensor 100 can be encapsulated in a flexible polymer such as topaz, parylene coating, or composite coatings of polymer-metal-polymer or other multiple layers of polymer and metal films (e.g., metals such as aluminum, titanium, etc.) to minimize vapor transport.

The pressure measurements (and/or the strain measurements or measurements indicating the amount that a surface bends, stretches or otherwise contorts) collected from the embodiments above can be used for applications such as: motor function disease detection and/or monitoring Parkinson's disease detection and/or monitoring, Huntington's disease detection and/or monitoring, etc. The measurements can also be used to determine medication efficacy of an individual as they consume a medication over a period of time. For example, if a patient is taking a defined medication to minimize body tremors, the manner in which the fingernail bends can be indicative of how well the body tremors are being managed. Thus, the strain on the strain sensor 100 can indicate the efficacy of the medication. In this manner, the strain sensor 100 can detect, monitor and/or facilitate detection or monitoring of certain diseases and correlate certain risks factors with the diseases.

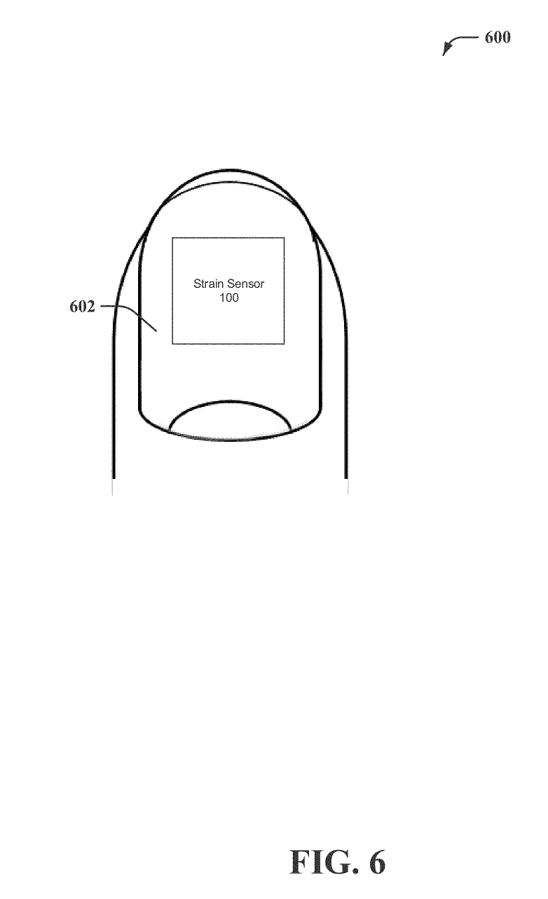

FIG. 6 illustrates a non-limiting top view 600 of the sensor in used in accordance with one or more embodiments described herein. Repetitive description of like elements employed in other embodiments described herein is omitted for sake of brevity.

The strain sensor 100 can be attached to the fingernail 602 via fingernail glue or another adhesive. In one embodiment, cyanoacrylate can be used to attach the strain sensor 100 to the fingernail 602. Cyanoacrylate contains ethyl cyanoacrylate, polymethyl methacrylate, butylated hydroxyanisole, butylparaben, propylparaben, tocopherol and retinyl palmitate in some embodiments. However, any number of other types of adhesive can also be used. Additionally, propylene carbonate can be used as the fingernail glue remover. Propylene carbonate is used as a polar, aprotic solvent. Propylene carbonate does not cause skin irritation or sensitization and comprises acetone, propylene carbonate, isopropyl alcohol, limonene, and alcohol.

FIG. 7A illustrates a non-limiting spectrum peak shift graph of wavelength shift per reflection percentage due to a strain on the integrated photonic crystal structure in accordance with one or more embodiments described herein. FIG. 7B illustrates a non-limiting spectrum peak shift graph of wavelength shift per transmission percentage due to a strain on the integrated photonic crystal structure in accordance with one or more embodiments described herein. Repetitive description of like elements employed in other embodiments described herein is omitted for sake of brevity.

In a peak shift graph, light intensity changes can be shown. Therefore, if a fixed wavelength serves as an input to the strain sensor 100, then a fixed intensity associated with the wavelength can be expected. However, an observed deviation from the fixed wavelength and intensity via the graphs of FIGS. 7A and 7B can indicate a change in strain experienced by the strain sensor 100.

Since a fingernail can change shape depending upon its orientation and use, the amount of strain or change in shape of the fingernail can correspond to how much stress or strain is being put on the strain sensor 100. For example, nanograting expansion and contraction can be observed in the peak shift associated with the light reflected from the nanogratings to indicate how much a fingernail has expanded or contracted. Thus, the nanogratings can expand and/or contract with the fingernail. Consequently, the integrated sensor system 500 can determine how much pressure the fingernail is applying to an object (e.g., table, coffee cup, keyboard) when the fingernail interacts with the object. As depicted in FIG. 7A, the peak of the reflection graph 700 can shift to the left, indicating lower wavelengths, when the strain sensor 100 is experiencing stress. Additionally, the peak of the transmission graph 702 in FIG. 7B can also shift to the left, indicating lower wavelengths, when the strain sensor 100 is experiencing stress.

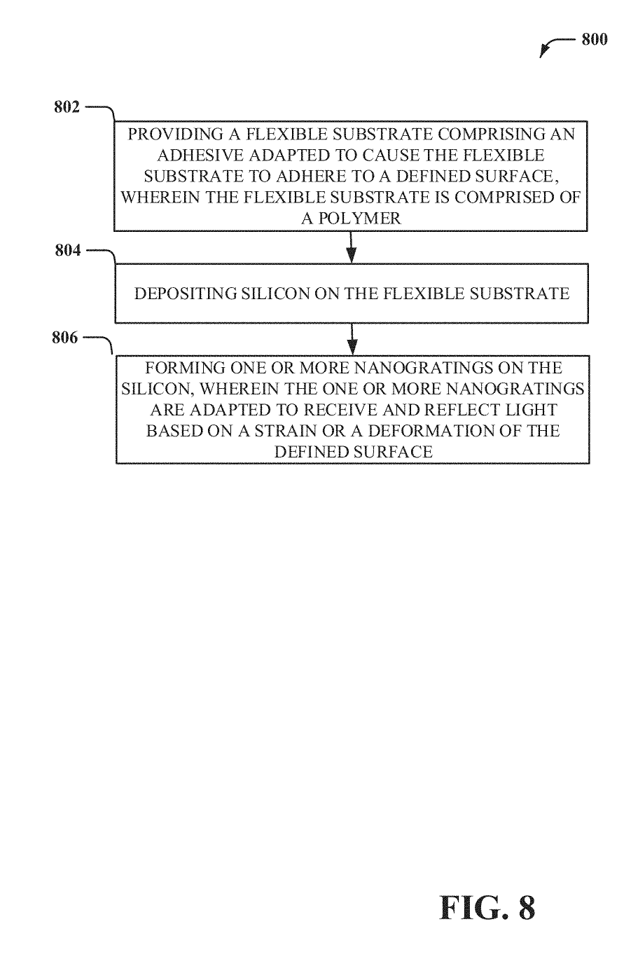

FIG. 8 illustrates a flow diagram of an example, non-limiting method 800 for fabricating an integrated sensor (e.g., integrated fingernail sensor) in accordance with one or more embodiments described herein. Repetitive description of like elements employed in other embodiments described herein is omitted for sake of brevity.

At 802, method 800 can comprise providing a flexible substrate comprising an adhesive adapted to cause the flexible substrate to adhere to a defined surface, wherein the flexible substrate is comprised of a polymer. In some embodiments, the flexible or polymer substrate can be a Si substrate attached to a fingernail employing cyanoacrylate. At 804, the method can comprise depositing silicon on the flexible substrate. Additionally, at 806 the method can comprise forming one or more nanogratings on the silicon, wherein the one or more nanogratings are adapted to receive and reflect light based on a strain or a deformation of the defined surface (e.g., fingernail 602). The nanogratings can be formed via the spalling process or temporary bonding-debonding process depicted in FIG. 2 or FIG. 3.

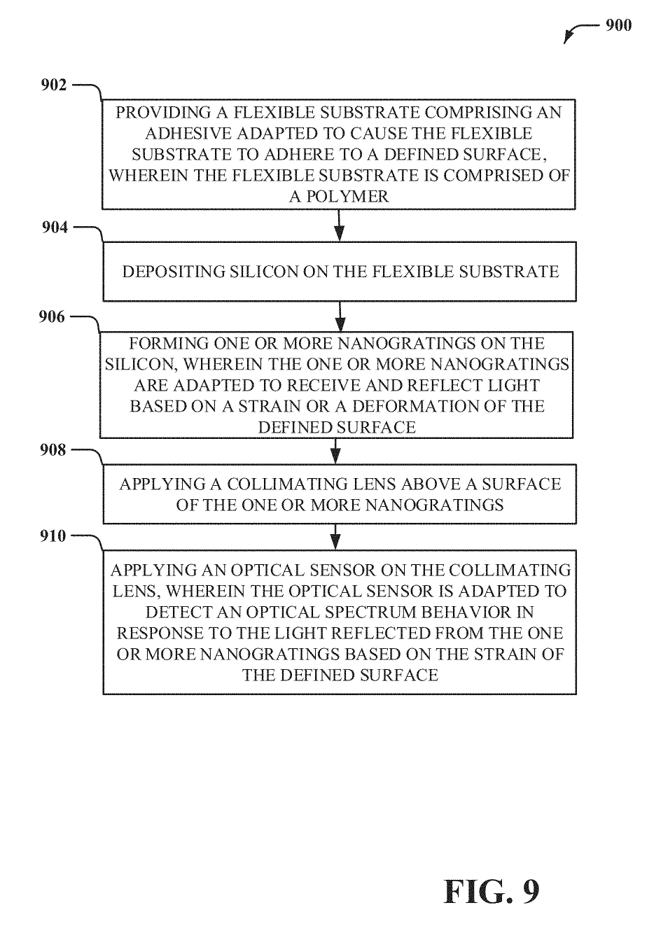

FIG. 9 illustrates a flow diagram of another example, non-limiting method for fabricating an integrated sensor (e.g., integrated fingernail sensor) in accordance with one or more embodiments described herein. Repetitive description of like elements employed in other embodiments described herein is omitted for sake of brevity.

In another embodiment, at 902, the method 900 can comprise providing a flexible substrate comprising an adhesive adapted to cause the flexible substrate to adhere to a defined surface, wherein the flexible substrate is comprised of a polymer. The flexible or polymer substrate can be a Si substrate attached to a fingernail with cyanoacrylate. At 904, the method can comprise depositing silicon on the flexible substrate. Additionally, at 906 the method can comprise forming one or more nanogratings on the silicon, wherein the one or more nanogratings are adapted to receive and reflect light based on a strain or a deformation of the defined surface (e.g., fingernail 602). The nanogratings can be formed via the spalling process or temporary bonding-debonding process depicted in FIG. 2 or FIG. 3. At 908, the method can further comprise applying a collimating lens (e.g., collimating lens 508) above a surface of the nanogratings (e.g., nanogratings 116, 118, 120). The collimating lens can receive light from a light source and reflect the light from the nanogratings to a spectrometer. Additionally, the method can comprise applying an optical sensor on collimating lens, wherein the optical sensor is adapted to detect an optical spectrum behavior in response to the light reflected from the one or more nanogratings based on the strain of the defined surface (e.g., fingernail 602).

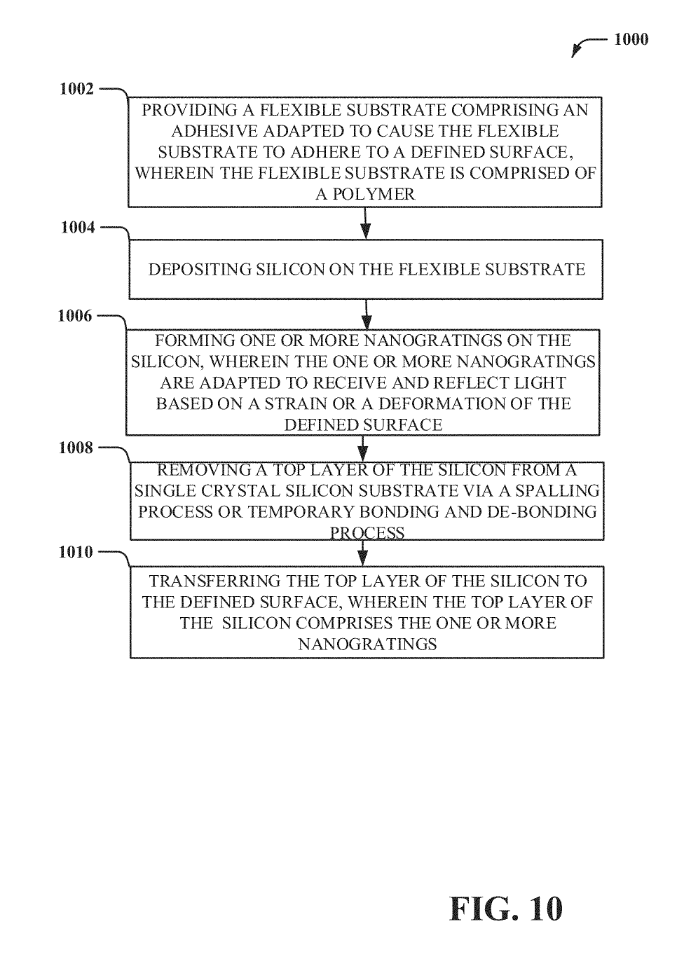

FIG. 10 illustrates a flow diagram of yet another example, non-limiting method for fabricating an integrated sensor (e.g., integrated fingernail sensor) in accordance with one or more embodiments described herein. Repetitive description of like elements employed in other embodiments described herein is omitted for sake of brevity.

In yet another embodiment, the method 1000 can comprise, at 1002, providing a flexible substrate comprising an adhesive adapted to cause the flexible substrate to adhere to a defined surface, wherein the flexible substrate is comprised of a polymer. The flexible or polymer substrate can be a Si substrate attached to a fingernail with cyanoacrylate. At 1004, the method can comprise depositing silicon on the flexible substrate. Additionally, at 1006, the method can comprise forming one or more nanogratings on the silicon, wherein the one or more nanogratings are adapted to receive and reflect light based on a strain or a deformation of the defined surface (e.g., fingernail 602). The nanogratings can be formed via the spalling process or a temporary bonding and de-bonding process depicted in FIG. 2 or FIG. 3. At 1008, the method can comprise removing a top layer of the silicon from a single crystal silicon substrate via a spalling process or temporary bonding and de-bonding process. Furthermore, at 1010, the method can comprise transferring the top layer of the silicon to the defined surface (e.g., fingernail 602), wherein the top layer of the silicon comprises the one or more nanogratings.

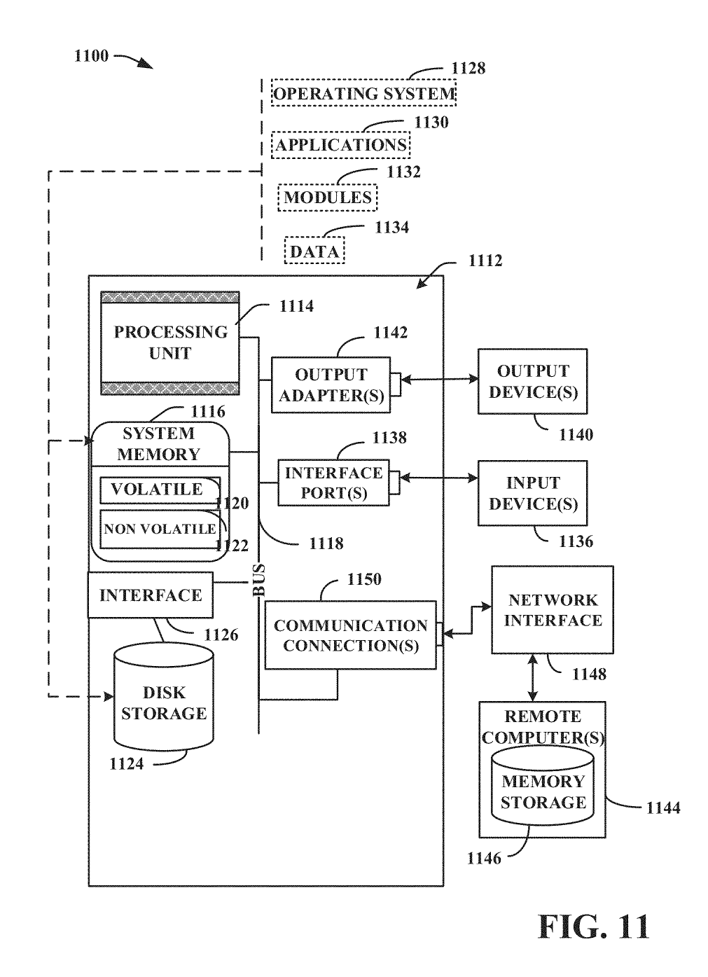

In order to provide a context for the various aspects of the disclosed subject matter, FIG. 11 as well as the following discussion is intended to provide a general description of a suitable environment in which the various aspects of the disclosed subject matter can be implemented. FIG. 11 illustrates a block diagram of an example, non-limiting operating environment in which one or more embodiments described herein can be facilitated. With reference to FIG. 11, a suitable operating environment 1100 for implementing various aspects of this disclosure can also include a computer 1112. The computer 1112 can also include a processing unit 1114, a system memory 1116, and a system bus 1118. The system bus 1118 couples system components including, but not limited to, the system memory 1111 to the processing unit 1114. The processing unit 1114 can be any of various available processors. Dual microprocessors and other multiprocessor architectures also can be employed as the processing unit 1114. The system bus 1118 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Firewire (IEEE 1394), and Small Computer Systems Interface (SCSI).

The system memory 1116 can also include volatile memory 1120 and nonvolatile memory 1122. The basic input/output system (BIOS), containing the basic routines to transfer information between elements within the computer 1112, such as during start-up, is stored in nonvolatile memory 1122. By way of illustration, and not limitation, nonvolatile memory 1122 can include read only memory (ROM), programmable ROM (PROM), electrically programmable ROM (EPROM), electrically erasable programmable ROM (EEPROM), flash memory, or nonvolatile random access memory (RAM) (e.g., ferroelectric RAM (FeRAM). Volatile memory 1120 can also include random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), Rambus dynamic RAM, and Phase Change Memory (PCM).

Computer 1112 can also include removable/non-removable, volatile/non-volatile computer storage media. FIG. 11 illustrates, for example, a disk storage 1124. Disk storage 1124 can also include, but is not limited to, devices like a magnetic disk drive, floppy disk drive, tape drive, Jaz drive, Zip drive, LS-160 drive, flash memory card, or memory stick. The disk storage 1124 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 1124 to the system bus 1118, a removable or non-removable interface is typically used, such as interface 1126. FIG. 11 also depicts software that acts as an intermediary between users and the basic computer resources described in the suitable operating environment 1100. Such software can also include, for example, an operating system 1128. Operating system 1128, which can be stored on disk storage 1124, acts to control and allocate resources of the computer 1112.

System applications 1130 take advantage of the management of resources by operating system 1128 through program modules 1132 and program data 1134, e.g., stored either in system memory 1116 or on disk storage 1124. It is to be appreciated that this disclosure can be implemented with various operating systems or combinations of operating systems. An entity enters commands or information into the computer 1112 through input device(s) 1136. Input devices 1136 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1114 through the system bus 1118 via interface port(s) 1138. Interface port(s) 1138 include, for example, a serial port, a parallel port, a game port, and a universal serial bus (USB). Output device(s) 1140 use some of the same type of ports as input device(s) 1136. Thus, for example, a USB port can be used to provide input to computer 1112, and to output information from computer 1112 to an output device 1140. Output adapter 1142 is provided to illustrate that there are some output devices 1140 like monitors, speakers, and printers, among other output devices 1140, which require special adapters. The output adapters 1142 include, by way of illustration and not limitation, video and sound cards that provide a means of connection between the output device 1140 and the system bus 1118. It should be noted that other devices and/or systems of devices provide both input and output capabilities such as remote computer(s) 1144.

Computer 1112 can operate in a networked environment using logical connections to one or more remote computers, such as remote computer(s) 1144. The remote computer(s) 1144 can be a computer, a server, a router, a network PC, a workstation, a microprocessor based appliance, a peer device or other common network node and the like, and typically can also include many or all of the elements described relative to computer 1112. For purposes of brevity, only a memory storage device 1146 is illustrated with remote computer(s) 1144. Remote computer(s) 1144 is logically connected to computer 1112 through a network interface 1148 and then physically connected via communication connection 1150. Network interface 1148 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL). Communication connection(s) 1150 refers to the hardware/software employed to connect the network interface 1148 to the system bus 1118. While communication connection 1150 is shown for illustrative clarity inside computer 1112, it can also be external to computer 1112. The hardware/software for connection to the network interface 1148 can also include, for exemplary purposes only, internal and external technologies such as, modems including regular telephone grade modems, cable modems and DSL modems, ISDN adapters, and Ethernet cards.

The present disclosure may be a system, a method, an apparatus and/or a computer program product at any possible technical detail level of integration. The computer program product can include a computer readable storage medium (or media) having computer readable program instructions thereon for causing a processor to carry out aspects of the present disclosure. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely or partly on a computer for an entity, as a stand-alone software package, and/or partly on a remote computer or entirely on the remote computer or server. In the latter scenario, the remote computer can be connected to the entity computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system comprising:
a flexible substrate comprising an adhesive adapted to cause the flexible substrate to adhere to a defined surface; and
a silicon substrate or film disposed on the flexible substrate, wherein the silicon substrate or film is formed to include one or more nanogratings adapted to receive and reflect light based on strain on the defined surface.

2. The system of claim 1, further comprising an optical sensor coupled to the one or more nanogratings and that detects a shift of an optical spectrum peak in response to the light reflected from the one or more nanogratings based on the strain on the defined surface.

3. The system of claim 2, wherein the optical sensor comprises a spectrometer coupled to the one or more nanogratings via an optical cable.

4. The system of claim 2, further comprising a transmitter coupled to the optical sensor and that transmits a signal indicative of information associated with the optical spectrum peak.

5. The system of claim 4, wherein the information comprises medical information regarding a health condition of a user to which the flexible substrate and the silicon substrate or film is applied.

6. The system of claim 1, wherein the defined surface comprises a surface within a group consisting of: a fingernail or skin of a user to which the flexible substrate and the silicon substrate or film are applied.

7. The system of claim 1, further comprising a mobile detector remote from the one or more nanogratings and that detects a shift of an optical spectrum peak in response to the light reflected from the one or more nanogratings based on the strain on the defined surface.

8. The system of claim 1, further comprising a collimating lens coupled to the one or more nanogratings and adapted to receive the light from a light source and reflect the light from the one or more nanogratings.

9. The system of claim 8, wherein the collimating lens is positioned above the one or more nanogratings.

10. A method, comprising:
providing a flexible substrate comprising an adhesive adapted to cause the flexible substrate to adhere to a defined surface, wherein the flexible substrate is comprised of a polymer;
depositing silicon on the flexible substrate; and
forming one or more nanogratings on the silicon, wherein the one or more nanogratings are adapted to receive and reflect light based on a strain or a deformation of the defined surface.

11. The method of claim 10, further comprising:
applying a collimating lens above a surface of the one or more nanogratings; and
applying an optical sensor on the collimating lens, wherein the optical sensor is adapted to detect an optical spectrum behavior in response to the light reflected from the one or more nanogratings based on the strain or the deformation of the defined surface.

12. The method of claim 10, wherein the forming the one or more nanogratings comprises forming the one or more nanogratings via a nano-printing step for a resist and etching process.

13. The method of claim 10, further comprising:
removing a top layer of the silicon from a single crystal silicon substrate via a spalling process or temporary bonding and de-bonding process; and
transferring the top layer of the silicon to the defined surface, wherein the top layer of the silicon comprises the one or more nanogratings.

14. The method of claim 10, wherein the forming the one or more nanogratings comprises forming the one or more nanogratings via a nano lithographic step and etching process.

15. The method of claim 10, wherein the flexible substrate is a silicon substrate, and wherein the forming the one or more nanogratings comprises:
depositing an adhesive layer on a first side of the flexible substrate;
depositing a release layer on the adhesive layer;
depositing a glass layer on the release layer;
reducing a thickness of the flexible substrate from a first thickness to a second thickness;
etching the flexible substrate, resulting in an etched silicon substrate;
depositing a polymer or an optical material on the etched silicon substrate;
utilizing the release layer to remove the glass layer;
removing the adhesive layer; and
removing the glass layer.

16. The method of claim 15, wherein the optical material is elected from a group consisting of: titanium dioxide, silicon oxide and glass that has a first optical property matching a second optical property of the polymer on the etched silicon substrate.

17. An apparatus comprising:
a polymer substrate comprising an adhesive adapted to cause the polymer substrate to adhere to a defined surface; and
a silicon substrate disposed on the polymer substrate, wherein the silicon substrate comprises one or more nanogratings adapted to receive and reflect light based on strain on the defined surface.

18. The apparatus of claim 17, further comprising:
a capacitive sensor coupled to the one or more nanogratings and that detects the strain on the defined surface, or a a photodetector sensor coupled to the one or more nanogratings and adapted to receive and reflect the light to detect the strain on the defined surface.

19. The apparatus of claim 17, further comprising:
a photoplethysmogram sensor coupled to the one or more nanogratings and adapted to measure a change in an absorption of the light by the defined surface.

20. The apparatus of claim 17, further comprising:
a temperature sensor coupled to the one or more nanogratings and adapted to measure a change in temperature by the defined surface.

21. A system comprising:
a flexible substrate comprising an adhesive adapted to cause the flexible substrate to adhere to a defined surface;
a silicon substrate or film disposed on the flexible substrate, wherein the silicon substrate or film is formed to include one or more nanogratings adapted to receive and reflect light based on strain on the defined surface; and
a mobile device configured to receive strain data associated with a strain experienced by the flexible substrate.

22. The system of claim 21, wherein the mobile device is further configured to receive the light that has been reflected from the one or more nanogratings.

23. The system of claim 21, wherein the strain data results in an optical spectrum peak shift represented on a display of the mobile device.

24. An apparatus comprising:
a polymer substrate comprising an adhesive adapted to cause the polymer substrate to adhere to a defined surface;
a silicon substrate disposed on the polymer substrate, wherein the silicon substrate comprises one or more nanogratings adapted to receive and reflect light based on strain on the defined surface, resulting in reflected light; and
a collimating lens adapted to receive the reflected light from the one or more nanogratings.

25. The apparatus of claim 24, further comprising:
an optical sensor adapted to detect an optical spectrum behavior in response to receiving the reflected light from the one or more nanogratings.
